(19) United States
(12) Patent Application Publication
(10) Pub. No.: US 2007/0042055 A1
(43) Pub. Date: Feb. 22, 2007

(54) Herbal formulations and cigarettes containing same useful in controlling body weight

(75) Inventors: Pushpangadan Palpu, Lucknow (IN); Chandana Venkateshwara Rao, Lucknow (IN); Ajay Kumar Singh Rawat, Lucknow (IN); Raghavan Govindarajan, Lucknow (IN); Sharad Kumar Srivastava, Lucknow (IN); Kuttanpillai Narayanan Nair, Lucknow (IN); Sayyada Khatoon, Lucknow (IN); Subha Rastogi, Lucknow (IN); Sanjeev Kumar Ojha, Lucknow (IN); Biswajit Deb, Mumbai (IN); Srinivas Subramanian, Mumbai (IN)

Correspondence Address: KENYON & KENYON LLP, 1500 K STREET N.W., SUITE 700, WASHINGTON, DC, 20005, US

(73) Assignees: Council of Scientific and Industrial Research, New Delhi (IN); Godfrey Phillips India Limited, Mumbai (IN)

(21) Appl. No.: 11/407,208

(22) Filed: Apr. 20, 2006

(30) Foreign Application Priority Data
Apr. 20, 2005 (IN) 0988/DEL/2005

(51) Int. Cl. A61K 36/736; A61K 36/53; A61K 36/8962; A61K 36/48; A61K 36/73; A61K 36/81; A61K 36/232; A61K 36/23

(52) U.S. Cl. 424/725; 424/751; 424/735; 424/754; 424/757; 424/745; 424/765

(57) ABSTRACT

The present invention relates to the an herbal formulation comprising a combination of fiber, powder, and/or granules of aerial parts of plants selected from Hemedesmus indicus, Acorus calamus, Murraya Koenigii, Alpinia galanga, Papaver somniferum, Amorphophallus campanulatus, Hygrophila auriculata, Hygrophila schulli, Cuminum cyminum, Apium graveolens, Colocasia esculenta. The present invention further relates bidis, cigarettes, cigars, pipes, snuffs, cigars, and chewing gums comprising the herbal composition.

### FIELD OF THE INVENTION

The present invention relates to a herbal formulation comprising a synergistic combination of fiber, powder, and/or granules of aerial parts of at least two plants selected from the group consisting of Hemedesmus indicus, Acorus calamus, Murraya Koenigii, Alpinia galanga, Papaver somniferum, Amorphophallus campanulatus, Hygrophila auriculata, Hygrophila schulli, Cuminum cyminum, Apium graveolens, and Colocasia esculenta.

More particularly, it relates to the use of said formulation in products such as bidis, cigarettes, cigars, snuffs, pipes and/or chewing gums.

### BACKGROUND OF THE INVENTION

Medicated smoke may become a part of the daily routine. When used as therapy, the smoke is inhaled through the nose for head and neck disease, and through the mouth for throat disorders. The potential advantages of delivering a drug to the lung by inhalation have been well known to physicians and drug abusers for years. For drugs that exert their biological effect in the lung, these include rapid onset of action, reduced dose and minimized side effects compared to the same drug delivered by mouth. First line asthma therapy is currently based on inhaled corticosteroids that minimize the problems associated with oral steroids, such as adrenal suppression, electrolyte imbalance and muscle weakness. Tobacco smoke has been used in western culture for more than 400 years; human inhalation of cigarette smoke is a twentieth century phenomenon with major medical and economic consequences. Excessive smoking is now recognized as one of the major health problems throughout the world. The most important difficulty results from the fact that heavy smokers are dependent on nicotine, which is considered to be one of the risk factors in tobacco smoke. During the combustion of tobacco, important risk factors are formed, such as carbon monoxide, tar products, aldehydes and hydrocyanic acid (U.S. Pat. No. 5,335,678).

U.S. Patent application 2004/0103908 describes an herbal cigarette free from tobacco, containing a predetermined mixture of different herbs packed in filtered or non-filtered cigarette paper tubes. U.S. Pat. No, 6,497,234 relates to a mixture of herbs and other natural products that can be used as a healthy substitute for tobacco, and also alleviates the addictive effects of nicotine, and provides an aid to quit smoking tobacco-related products. U.S. Patent Application 2005/0061828 relates generally to the field of packaging machines, which provides for the packaging of at least two types of smoking products consisting of tobacco cigarettes/cigars and bidis. There remains a need for functional cigarettes useful for controlling body weight. The present invention meets this need as it provides herbal formulations and herbal cigarette useful for controlling body weight. Further, the herbal formulations provide additional benefits in innumerable ways to the health of the body and mind.

### SUMMARY OF THE INVENTION

One embodiment of the invention provides an herbal formulation comprising a fiber, powder, and/or granules of the aerial parts of at least two plants selected from the group consisting of Hemedesmus indicus, Acorus calamus, Murraya Koenigii, Alpinia galanga, Papaver somniferum, Amorphophallus campanulatus, Hygrophila auriculata, Hygrophila schulli, Cuminum cyminum, Apium graveolens, and Colocasia esculenta. Preferably the herbal formulation further comprises one or more flavoring agents such as essential and/or aromatic oils.

Another embodiment of the present invention provides the use of the said herbal formulation in products such as, but not limited to, bidis, cigarettes, cigars, pipes, snuffs and chewing gums.

Another embodiment of the present invention provides a novel herbal cigarette comprising the formulation described herein, useful for controlling body weight and change in body weight composition.

The invention also provides for methods of (1) controlling weight or reducing weight, and/or suppressing the appetite; (2) treating or relieving asthma or respiratory diseases; (3) improving digestion and evacuation of bowel; (4) spreading a good aroma to the surrounding air; (5) relieving stress; (6) and reducing the levels of bad fat content by providing an herbal formulation of the present invention.

### DETAILED DESCRIPTION OF THE PRESENT INVENTION

The present invention provides an herbal formulation comprising a fiber, powder and/or granules of aerial parts of at least two plants selected from the group consisting of Hemedesmus indicus, Acorus calamus, Murraya Koenigii, Alpinia galanga, Papaver somniferum, Amorphophallus campanulatus, Hygrophila auriculata, Hygrophila schulli, Cuminum cyminum, Apium graveolens, and Colocasia esculenta. It is believed that a combination of at least two of the above listed plants provides a synergistic combination. Aerial parts of plants refers to the above ground parts of plants, for example, flowers, leaves and stems. The herbal formulation preferably also comprises one or more flavoring agents such as essential and/or aromatic oils. Essential oils are know in the art and is a common term for a volatile oil, usually having the characteristic odor or flavor of the plant from which it is obtained. Essential oils are often used to make perfumes and flavorings. Aromatic oils are known in the art and are also called fragrance oils, aroma oils, and flavor oils. Aromatic oils are often blended synthetic aroma compounds or natural essential oils that are diluted with a carrier like propylene glycol, vegetable oil, or mineral oil. Aromatic oils are used in perfumery, cosmetics, flavoring of food, and in aromatherapy.

In another embodiment of the present invention, the herbal formulation comprises a fiber, powder, and/or granules of aerial parts of plants comprising 2-8% Hemedesmus indicus, 0.05-1.2% Acorus calamus, 1-8% Murraya Koenigii, 2-15% Alpinia galangal, 0.5-13.5% Papaver somniferum, 2.5-7.5% Amorphophallus campanulatus, 1- 15% Hygrophila auriculata, 0.5%-7.8% Hygrophila schulli, 2-3% Cuminum cyminum, 3-5% Apium graveolens and 2-5% Colocasia esculenta. The formulation may further comprise one or more flavoring agents such as essential and/or aromatic oils.

Further, in one embodiment of the present invention, essential and/or aromatic oils may be, but are not limited to, clove, eucalyptus, Angelica archangel, Apium graveolens, Cananga odorata, Carum carvi, Citrus aurantifolia, Citrus limonum, Cymbopogon citrates, Cymbopogon martinii, Cinnamomum verum, Lavandula angustifolia, Lavendula officinalis, Melaleuca alternifolia, Myristicafragrans, Pelargonium graveolens, Rosa damascene, Santalum album, Jasminum multiflorum, Jasminum sambac, Ocimum sanctum, Ocimum gratissimum, Allium cepa, Allium sativum and Nicotiana tabacum.

In another embodiment of the present invention, there is provided a composition comprising herbal formulations of the present invention present up to about 50% of the total composition, and wherein the remainder of the composition comprises conventional additives, including tobacco. Conventional additives are known in the art. Conventional additives to bidis, cigarettes, cigars, pipes, snuff and chewing gum are also known in the art.

The herbal formulations of the present invention may be used to reduce weight, removing depression, relieve stress, spread fragrance or good aroma into the surrounding area, curing or relieving asthma or breathing difficulties, improving digestion and evacuation of bowel, and scavenging free radicals (by acting as a potent antioxidant). The formulations of the present invention also help in improving the potent enzymatic superoxide dismutase enhancing activity. The formulations also help in defending the body against the effects of a stressful environment, and provides vitality and energy. The herbal formulations of the present invention are non-irritating and have no toxic- effects. The herbal formulations do not induce itching, breathing problems, bronchial irritation, and pain in nose, ear and throat. Accordingly, the present invention also provides methods of reducing weight, removing/reducing depression, relieving stress, spreading fragrance or good aroma in surrounding area, curing or relieving asthma or breathing difficulties, improving digestion and evacuation of bowel, and scavenging free radicals (by acting as a potent antioxidant); and improving the potent enzymatic superoxide dismutase enhancing activity by providing the herbal formulations of the present invention. The herbal formulations may be provided to a subject in the form of bidis, cigars, cigarettes, pipes, snuff, chewing gum and the like, which have been formulated with the herbal formulation or compositions of the present invention.

The present invention also provides bidis, cigars, cigarettes, pipes, snuff and chewing gum, and the like, comprising the herbal formulations of the present invention. In another embodiment, the invention provides bidis, cigars, cigarettes, pipes, snuff and chewing gum comprising the herbal formulations and further comprises essential and/or aromatic oils, and/or conventional additives, and/or tobacco. Since these products have less tobacco than conventional bidis, cigars, cigarettes, etc., (as they also contain herbal formulations of the present invention), they also provide the additive benefit of having or producing less tar than the conventional tobacco products.

The plants used in the preparation of the herbal formulations and compositions of the present invention have been reported to have following descriptive characteristics.

Hemedesmus indicus Family: Asclepiadaceae

Botanical Description—A slender, twining or prostate perennial shrub with cylindrical stems, thickened at the nodes, and aromatic roots. Leaves simple, opposite or whorled, short-petioled, variable in shape from broadly ovate to oblong-elliptic, linear or linear-lanceolate, obtuse or apiculate, 3-10 cm long and 0.3-3.8 cm wide, base acute, rounded or truncate, apex rounded or emarginate and apiculate, leathery, dark green above, often variegated with white along the veins, paler beneath, leaves on flowering shoots broader and brighter green than other leaves; petioles 0.1-0.6 cm long. Flowers small, greenish yellow outside and purplish inside, crowded in subsessile axillary cymes shorter than the leaves; calyx deeply 5-lobed, glandular within; corolla 5-partite, 0.6-0.8 cm in diameter when expanded, lobes thick, ovate-oblong. Fruit (follicle) glabrous, often purplish, slender, cylindrical, sometimes curved, 10-12 cm long and 0.5-0.6 cm wide, divaricate, containing numerous black, flattened, ovate-oblong seeds with a silvery—white coma. Flowers mainly between July and October and fruits from October to February within its Indian range.

Distribution and habitat—Common in open deciduous and scrub forests, hedges and on degraded sites from the upper Gangetic plains of northern India eastwards to Assam and southwards through peninsular India to Sri Lanka.

Medicinal properties and uses—The dried roots, known commercially as Hemedesmus or Anantamul or Indian Sarsaparilla, are official in the Indian pharmacopoeia. The root and root bark are considered tonic, alternative, demulcent, diaphoretic, diuretic and blood purifying. They are used in several well known ayurvedic preparations for treating bowel complaints, elephantiasis, fever, nausea, and vomiting. Administered in powder form or as an infusion, decoction or syrup, it is prescribed for fever, leucorrhoea, chronic rheumatism and skin diseases. The root paste is applied to the forehead to reduce fever among the Paharia in southern Bihar. Among the Kondhs of south western Orissa, the crushed root is applied externally to relieve itching and its decoction is used to control excessive menstrual flow, or mixed with mother's milk to check diarrhea in infants. A hot infusion of the root bark with milk and sugar is given to children to relieve chronic cough and diarrhea. In Kerela, the milky latex is used to relieve eye inflammation.

Acorus calamus Family: Araceae

Botanical Description—A strongly aromatic semi-aquatic perennial herb; rhizomes creeping, jointed, somewhat vertical compressed, 1.3-2.5 cm thick, pale to dark brown and spongy inside. Leaves narrow up to 80 cm long, linear to narrowly ensi form, glossy bright green, apex acute, base amplexicaul; petioles sheathing for 20-50 cm, flowers pale green, fragrant, arranged compactly on a sessile, cylindrical, stumpy spadix 5-7 cm long. Fruits (berries) green, angular, 3-celled, fleshy, containing 1-3 oblong seeds. It is commonly known as sweet flag, flag root or sweet calomel.

Distribution and Habitat—Native to Europe, cultivated and sometimes naturalized throughout India, Myanmar (Burma) and Sri Lanka. Common in moist or marshy habitats between 300 and 2000 m elevation in the Himalayas.

Medicinal properties and uses—The dried rhizomes comprise the commercial drug calamus, used in Europe since the time of Hippocrates. In Ayurveda, they constitute the drug Vaca, considered to have antispasmodic, carminative and anthelmintic properties and to be capable of improving memory and intellect. It is used in various forms (as a powder, balm, enema or pills) to treat a wide variety of diseases and conditions, including epilepsy and other mental ailments, chronic diarrhea and dysentery, bronchial caratacts, intermittent fevers and glandular and abdominal tumors, liver and kidney ailments, rheumatism and eczema. The rhizomes are an ingredient in many compound ayurvedic and other traditional medicinal preparations. The rhizomes, roots and leaves yield a light brown to brownish-yellow volatile oil known as calamus oil which has been, credited with carminative, antispasmodic and antibacterial properties. Among the Gonds of Uttar Pradesh, the roots and root bark, taken together with black pepper, is reportedly used as an antidote for snakebite.

Murraya Koenigii Family: Rutaceae

Botanical Description—A large shrub or small evergreen or deciduous tree to 5 m tall with a short trunk, thin, smooth, grey or brown bark and a dense, shady crown. Most parts of the plant are covered with fine down and have a strong, peculiar smell. Leaves pinnately compound, 15-30 cm long, arranged spirally, crowded on the ends of branchlets, rachis terete, pubescent, petioles 1.2-1.5 cm long; leaflets 11-25, alternate on rachis, 2.5-3.5 cm long, ovate-lanceolate with an oblique base, margins irregularly crenate, pubescent beneath, petioles 2-3 mm long. Flowers white, 0.8-1 cm long, numerous in broad terminal clusters, calyx pubescent, lobes triangular, subacute; petal 6 mm long, linear-oblong, rounded at apex, gland-dotted. Fruits ovoid to subglobose, wrinkled or rough with glands, to 2.5 cm long and 0.8 cm in diameter, purplish-black when ripe; 2-seeded. Flowers mainly between February and April, and fruits between April and August, depending on locality. It is also commonly known as curry-leaf tree.

Distribution and habitat—Native to tropical Asia from the Himalayan foothills of India to Srilanka eastwards through Myanmar, Indochina, Southern China and Hainan. Frequently cultivated throughout India and elsewhere in the tropics of its leaves, used to flavor curries; sometimes naturalized outside its native range.

Medicinal properties and uses—The plant is highly valued for its leaves, an important ingredient in Indian cuisine, to promote appetite and digestion. In Ayurveda, they enter into several important compound preparations, and are considered useful for destroying pathogenic organisms and poisons, and treating skin diseases, worm, infestation and neurosis. The raw green leaves or their decoction are taken orally to treat dysentery and as a febrifuge. Crushed leaves are applied externally to cure skin eruptions and to relieve burns. The bark and roots are used as stimulants, and are applied externally to cure skin eruptions and to treat the bites of poisonous animals.

Hygrophila auriculata Family: Acanthaceae

Botanical Description—A stout herb up to 1.5 m high, more or less hispid with long hairs; stems numerous, erect, usually unbranched, subquadangular, thickened at the nodes. Leaves in whorls of 6, the outer 2 the largest, oblong-lancoelate or oblanceolate, up to 18 cm long and 3.2 cm wide, with sharp, yellow, axillary spines; the two outer leaves of each whorl larger and others much smaller. Flowers bluish-purple, in sessile axillary whorls; calyx 4-partite, lobes unequal, clothed with soft hairs; corolla distinctly 2-lipped, the upper2-lobed, the lower 3-lobed. Fruits 0.8 cm long, linear-oblong, pointed, containing 4-8 orbicular seeds. Flowers between October and December in central Indian deciduous forest regions.

Distribution and habitat—Throughout the plains of India, in moist habitats such as marshy margins of canals and rice fields. Also found in tropical Himalayas, Myanmar, Indonesia and Malaysia.

Medicinal properties and uses—In Ayurveda, seeds and roots comprise the drug Kolilaksah, a reputed remedy for arthritis. The bitter seeds are considered an aphrodisiac and tonic and are used as a uterine sedative for pregnant women, and for treating diseases of biliousness. The seeds are given with sugar or milk in the treatment of impotence, gonorrhea and spermatorrhea. A paste of the seeds mixed with buttermilk or whey, is given for diarrhea. A decoction of the roots is used as a diuretic and to treat rheumatism, gonorrhea, and other diseases of the genito-urinary tract, jaundice and anasarca. The leaves are considered oleaginous, tonic, aphrodisiac and hypnotic; they are used to treat diarrhea, dysentery, thirst, urinary calculi and urinary discharges, inflammations, constipation, abdominal troubles. Among the rural inhabitant of Bhavnagar district of Gujarat, the whole plant is reportedly used to treat jaundice, dropsy, and rheumatism. In Unani system, the plant is known as Talamakhana, the leaves being considered useful for relieving cough; they are applied externally to treat gleet, lumbago and joint pains. In Siddha, the flower, seeds and whole plant are used for treating anemia, dropsy, ascites, constipation, piles and aphrodisiac.

Alpinia galanga Family: Zingeberaceae

Botanical Description: Dried rhizomes constitute greater galangal. Used in rheumatism and bronchial catarrh; also considered stimulant and carminative. Rhizome yields an essential oil, used in perfumery as a source of methyl cinnamate and cineol. They are used for pickling; as condiment, and for seasoning fish; also substituted for ginger.

Medicinal properties and uses: Flowers eaten raw or pickled. Herb accredited with antitubercular properties. Seeds used for colic, diarrhea and vomiting, and as a sternutatory.

Papaver somniferum Family: Papaveraceae

Part used: Leaves

Botanical Description: Cultivated for production of opium which is harvested by incising the capsules at a particular stage known as “industrial maturity;” the product of first lancing contains higher percentage of morphine. Opium used as a narcotic, sedative, anodyne, antispasmodic, hypnotic, and sudorific. Whole opium is much less used, its pure alkaloids, morphine and codeine and their salts are preferred.

Medicinal properties and uses: Eating of opium creates a sense of euphoria and is habit forming; consumed orally, by smoking, and by injection. Poppy seed oil used for culinary purposes, free from narcotic action; also used in diarrhea and dysentery and in compositions for skin care. Capsules contain the same constituents as opium; infusion applied as a soothing application. Extract used as a sedative against irritating cough.

Amorphophallus campanulatus Family: Araceae

Botanical description: A tuberous, stout, indigenous herb, 1.0-1.5 m found almost throughout India. Tubers depressed, globose or hemispherical, dark brown out side, pale dull brown, sometimes almost white, with numerous long roots; leaves solitary, tripartite, 30-90 cm broad. It is also commonly known as elephant yam.

Medicinal uses: The corms are irritant due to the presence of calcium oxalate. It can also be made into pickles. The stems can be used as cattle feed. They are rich in nutrients and minerals. They are carminative, aperient and expectorant. The fresh corms are an acrid stimulant and expectorant and increase appetite and taste. They are applied to treat acute rheumatism. They are also used in dysentery, piles and hemorrhoids.

Phytochemistry: It is nutritious and food values compares well with other tubers. It contains proteins, fat, fiber, carbohydrates, starch, oxalic acid and minerals calcium, phosphorus, Iron and vitamin A. Besides these, glucose, galactose and xylose are also present. The presence of an active diastatic enzyme is reported. The corm contains betulinic acid, beta sitosterol, stigmasterol and beta sitosterol palmitate.

Pharmacology: The methanolic extract of the corms showed significant effect on the uterus of the guinea pig. The fermented juice of the petioles is used to cure diarrhea. The seeds are also applied externally as irritant in treating rheumatic swelling.

Hygrophila auriculata Family: Acanthaceae

Botanical description: A stout herb up to 1.5 m high, more or less hispid with long hairs; stems numerous, erect, usually unbranched, subquadragnular thickened at the nodes. Leaves in whorls of 6, the outer 2 the largest, oblong-lanceolate or oblanceolate, up to 18 cm long and 3.2 cm wide, with sharp yellow, axillary spines; the two outer leaves of each whorl larger and others much smaller.

Medicinal uses: In Ayurveda, seeds and roots comprise the drug kolilakash, reputed remedies for arthritis. The bitter seeds are considered aphrodisiac and tonic and used as a uterine sedative for pregnant women and for treating diseases of the blood and biliousness. The seeds are given with sugar or milk in the treatment of impotence, gonorrhea and spermatorrhoea. A paste of seeds, mixed with buttermilk is given for diarrhea. A decoction of the root is used as a diuretic and to treat rheumatism. The leaves are considered oleaginous, tonic, aphrodisiac and hypnotic.

Hygrophila schulli Family: Acanthaceae

Botanical description: A stout herb up to 1.5 m high, more or less hispid with long hairs; stems numerous, erect, usually unbranched, subquadragnular thickened at the nodes. Leaves in whorls of 6, the outer 2 the largest, oblong-lanceolate or oblanceolate, up to 18 cm long and 3.2 cm wide, with sharp yellow, axillary spines; the two outer leaves of each whorl larger and others much smaller.

Medicinal uses: In Ayurveda, seeds and roots comprise the drug kolilakash, reputed remedies for arthritis. The bitter seeds are considered aphrodisiac and tonic and used as a uterine sedative for pregnant women and for treating diseases of the blood and biliousness. The seeds are given with sugar or milk in the treatment of impotence, gonorrhea and spermatorrhoea. A paste of seeds, mixed with buttermilk is given for diarrhea. A decoction of the root is used as a diuretic and to treat rheumatism. The leaves are considered oleaginous, tonic, aphrodisiac and hypnotic. They are used to treat diarrhea, dysentery, thirst, urinary calculi and urinary discharges, diseases of the eyes, pains, ascites and abdominal troubles, anemia and constipation.

Colocasia esculenta Family: Araceae

Medicinal properties and uses: Tubers consumed boiled or fried. Young leaves eaten like spinach, also consumed when bleached. Juice of the petioles used as an astringent and styptic. This is also known as taro. Taro mucilage may be used as a size for impermeable paper. Tubers may be used for production of industrial alcohol. Taro flour can be used for soups and gruels, gravies and puddings. Taro-malt, prepared from flour, is good foods for infants and invalids.

Cuminum cyminum Family: Umbelliferae/Apiaceae

Medicinal properties and uses: Fruits used as a condiment in curry powders and for flavoring dishes. They are considered carminative, stomachic and astringent, useful in dyspepsia and diarrhea. Yield an essential oil used for flavoring liqueurs and cordials, also in perfumery. Besides the volatile oil, fruits contain also fixed oil with strong aromatic flavor. Commonly known as cumin.

Apium graveolens Family: Umbelliferae/Apiaceae

Medicinal properties and uses: Leaves used in salads and soups. Cremocarps (fruits) are used as spice. They are stimulant, carminative, sedative and nervine tonic, decoction used in rheumatism. Fruits yield an essential oil, used as a spasmodic and nervine stimulant. It probably acts as an intestinal antiseptic and has been found useful in rheumatoid arthritis. Roots are used as diuretic. Commonly referred to as wild celery.

Allium cepa Family: Alliaceae

Botanical Description: Commonly known as onion. Onions are a popular vegetable, also used for flavoring and pickling. Bulbs as well as fresh herb yield an essential oil.

Medicinal properties and uses: Onions considered stimulant, diuretic and expectorant, used against flatulence and dysentery. Roasted onions are applied as poultice.

Allium sativum Family: Alliaceae

Botanical Description: Bulbs consisting of cloves, used as a spice and condiment. Also known as garlic.

Medicinal Properties and uses: Preparations of garlic are used in pulmonary phthisis, gangrene of the lungs, and whooping cough. Laryngeal tuberculosis lupus, and duodenal ulcers are treated by garlic juices. Cloves also used for flatulence, colic, and atonic dyspepsia. Juice is applied in skin troubles and used as eardrops, it is used diluted with water as a vulnerary.

The following examples are given by way of illustration of the present invention and should not be construed to limit the scope of present invention.

### EXAMPLES

### Example 1

### Formulations

| | |
|---|---|
| | Percentage |
| Plant name | composition |

F1

| | |
|---|---|
| Nicotiana tabacum | 50 |
| Hemedesmus indicus | 15 |
| Acorus calamus | 10 |
| Murraya koenigii | 15 |
| Alpinia galanga | 10 |
| Flavoring agent | Quantity |
| | sufficient |

F2

| | |
|---|---|
| Nicotiana tabacum | 50 |
| Papaver somniferum | 10 |
| Amorphophallus | 15 |
| campanulatus | |
| Hygrophila auriculata | 10 |
| Murraya koenigii | 15 |
| Flavoring agent | Quantity |
| | Sufficient |

F3

| | |
|---|---|
| Nicotiana tabacum | 50 |
| Cuminum cyminum | 10 |
| Apium graveolens | 15 |
| Colocasia esculenta | 5 |
| Murraya koenigii | 15 |
| Hygrophila schulli | 5 |
| Flavoring agent | Quantity |
| | sufficient |

### Example 2

Blood Pressure Recording

Blood pressure (Systolic and diastolic) and pulse rate were recorded after 1^{st} and 2^{nd} week smoke exposure (smoke of formulations of the present invention) using a tail cuff blood pressure recorder (UGO Basile, Model no 58500; Comerio-varese, Italy). Rats were acclimatized to heating chamber (29-30° C.) for 30 min before recording the blood pressure (between 8:30 and 10:30 AM), in order to cause a sufficient vasodilatation in the caudal artery. Three recordings were measured for each rat and the average was calculated.

TABLE 1
Body weight, systolic, diastolic pressure
and pulse rate of smoke exposure in rats

| | Control rats | 1^{st} week | 2nd week |
|---|---|---|---|
| Body weight (g) | 320 ± 2.6 | 310 ± 2.8 | 297 ± 3.0 |
| Systolic blood | 117.6 ± 1.23 | 117.8 ± 1.24 | 118.1 ± 1.24 |
| pressure (mm Hg) | | | |
| Diastolic blood | 78.35 ± 1.24 | 79.15 ± 1.20 | 80.31 ± 1.25 |
| pressure (mm Hg) | | | |
| Pulse rate (beats/min) | 398.78 ± 27.5 | 399.89 ± 28.9 | 410.01 ± 30.8 |

These results show a change in the vital parameters of blood pressure and pulse rate. Both blood pressure and pulse rate increased. The body weight of the rats decreased. This may be due to the reduction in the intake of the food during the course of exposure of the smoke.

TABLE 2
Body weight, systolic, diastolic pressure and
pulse rate of smoke exposure in rats

| | Control rats | 1^{st} week | 2nd week |
|---|---|---|---|
| Body weight (g) | 325 ± 2.8 | 316 ± 3.4 | 307 ± 3.3 |
| Systolic blood pressure | 116.8 ± 1.24 | 119.5 ± 1.20 | 117.8 ± 1.22 |
| (mm Hg) | | | |
| Diastolic blood pressure | 77.87 ± 1.05 | 78.77 ± 1.26 | 83.30 ± 1.31 |
| (mm Hg) | | | |
| Pulse rate (beats/min) | 387.0 ± 25.3 | 397.90 ± 24.8 | 411.2 ± 26.5 |

These results show a change in the vital parameters on the blood pressure and pulse rate. Both blood pressure and pulse rate increased. However, there is a tendency in reduction of the body weight. This may be due to the reduction in the intake of the food during the course of exposure of the smoke.

1. An herbal formulation comprising a fiber, powder, and/or granules of aerial parts of at least two plants selected from the group consisting of Hemedesmus indicus, Acorus calamus, Murraya Koenigii, Alpinia galanga, Papaver somniferum, Amorphophallus campanulatus, Hygrophila auriculata, Hygrophila schulli, Cuminum cyminum, Apium graveolens, and Colocasia esculenta.

2. The herbal formulation of claim 1 further comprising a flavoring agent comprising an essential and/or aromatic oil.

3. A composition comprising an herbal formulation comprising a combination of fiber, powder, and/or granules of aerial parts of at least two plants selected from the group consisting of Hemedesmus indicus, Acorus calamus, Murraya Koenigii, Alpinia galanga, Papaver somniferum, Amorphophallus campanulatus, Hygrophila auriculata, Hygrophila schulli, Cuminum cyminum, Apium graveolens, Colocasia esculenta, wherein the herbal formulation comprises up to about 50% of the total composition, and wherein the composition further comprises tobacco.

4. The herbal formulation of claim 1, comprising 2-8% Hemedesmus indicus, 0.05-1.2% Acorus calamus, 1-8% Murraya Koenigii, 2-15% Alpinia galangal, 0.5-13.5% Papaver somniferum, 2.5-7.5% Amorphophallus campanulatus, 1-15% Hygrophila auriculata, 0.5%-7.8% Hygrophila schulli, 2-3% Cuminum cyminum, 3-5% Apium graveolens and 2-5% Colocasia esculenta.

5. A composition comprising the herbal formulation of claim 1, wherein the herbal formulation comprises up to about 50% of the total composition, and wherein the composition further comprises tobacco.

6. The herbal formulation as claimed in claim 1, wherein the essential and/or aromatic oils are selected from the group consisting of clove, eucalyptus, Angelica archangel, Apium graveolens, Cananga odorata, Carum carvi, Citrus aurantifolia, Citrus limonum, Cymbopogon citrates, Cymbopogon martinii, Cinnamomum verum, Lavandula angustifolia, Lavendula officinalis, Melaleuca alternifolia, Myristicafragrans, Pelargonium graveolens, Rosa damascene, Santalum album, Jasminum multiflorum, Jasminum sambac, Ocimum sanctum, Ocimum gratissimum, Allium cepa, Allium sativum and Nicotiana tabacum.

7. Bidis, cigarettes, cigars, pipes, snuffs, and chewing gums comprising the herbal formulation of claim 1.

8. Bidis, cigarettes, cigars, pipes, snuffs, and chewing gums comprising the herbal formulation of claim 3.

9. Bidis, cigarettes, cigars, pipes, snuffs, and chewing gums comprising the herbal formulation of claim 4.

10. Bidis, cigarettes, cigars, pipes, snuffs, and chewing gums comprising the herbal formulation of claim 5.

11. A method of suppressing appetite in a subject comprising providing to said subject, the bidis, cigarettes, cigars, pipes, snuffs, or chewing gums of claim 7.

12. A method of relieving asthma in a subject comprising providing to said subject, the bidis, cigarettes, cigars, pipes, snuffs, or chewing gums of claim 7.

13. A method of improving digestion in a subject comprising providing to said subject, the bidis, cigarettes, cigars, pipes, snuffs, or chewing gums of claim 7.

14. A method of scavenging free radicals in a subject comprising providing to said subject, the bidis, cigarettes, cigars, pipes, snuffs, or chewing gums of claim 7.
